SEQUENCE LISTING (4 sequences)

SEQ ID NO: 1 - DNA, 22 bp, Artificial sequence
  Synthetic
gaaataaaataaaaataaaata

SEQ ID NO: 2 - DNA, 22 bp, Artificial sequence
  Synthetic
tattttatttttattttatttc

SEQ ID NO: 3 - DNA, 26 bp, Artificial sequence
  Synthetic
gctgatcaaaaaacggaattggaccc

SEQ ID NO: 4 - DNA, 26 bp, Artificial sequence
  Synthetic
gggtccaattccgttttttgatcagc

(19) United States
(12) Patent Application Publication
(10) Pub. No.: US 2016/0222434 A1
(43) Pub. Date: Aug. 4, 2016

(54) Modified Polymerase Compositions, Methods and Kits

(71) Applicant: BRANDEIS UNIVERSITY, Waltham, MA (US)

(72) Inventors: Arthur H. Reis, Arlington, MA (US); Vincent Mecozzi, (US); Andrew Daab, (US); Lawrence J. Wangh, Auburndale, MA (US)

(73) Assignee: Brandeis University, Waltham, MA (US)

(21) Appl. No.: 15/021,843

(22) PCT Filed: Sep. 12, 2014
(86) PCT No.: PCT/US14/55348

§ 371 Date: Mar. 14, 2016

Related U.S. Application Data
(60) Provisional application No. 61/877,384, filed on Sep. 13, 2013.

(51) Int. Cl. C12Q 1/68

(52) U.S. Cl. CPC C12Q 1/686

(57) ABSTRACT

This invention relates to Type I DNA polymerases, for example, Taq DNA polymerase, and to methods and reagent kits utilizing such polymerases.

### FIELD OF THE INVENTION

This invention relates to Type I DNA polymerases, for example, Taq DNA polymerase, and to methods and reagent kits utilizing such polymerases.

### BACKGROUND

Type I DNA polymerases, for example Taq DNA polymerase, are well known, as is their use in biochemical reactions, particularly DNA amplification reactions such as the polymerase chain reaction (PCR), and detection assay methods that include amplification. In methods that include amplification reactions, polymerase specificity is an important and at times a limiting feature. Schemes to improve specificity include “hot start” polymerases, for example, antibody-bound enzyme that is inactive until heated to high temperature, thereby avoiding mispriming during preparation of amplification reaction mixtures at room temperature. Additionally, various primer designs reportedly improve selectivity. Our laboratory has described certain categories of reagent additives that, when included in amplification reactions, for example, PCR reactions, increase polymerase selectivity. See WO 2006/044995, WO 2010/105074, and U.S. Provisional Patent Application No. 61/755,822, filed 23 Jan. 2013. There remains a need for Type I DNA polymerases that inherently have improved selectivity in the absence of separate specificity-improving reagents.

### DEFINITIONS

“PCR”, as used herein, refers to the well-known nucleic acid amplification method known as the polymerase chain reaction. This invention applies to PCR methods generally, including, for example, symmetric PCR methods and non-symmetric PCR methods such as asymmetric PCR and LATE-PCR. Reverse transcription can be included (RT-PCR), if the target is RNA. PCR methods may include detection of amplification products, for example, by binding dye such as SYBR Green, that fluoresces when in contact with double-stranded (ds) DNA, and oligonucleotide probes whose hybridization to amplified target leads to a detectable signal, for example, a fluorescent signal.

As used herein, “non-symmetric PCR” means a PCR amplification in which one primer (the Limiting Primer) of a PCR primer pair is included in the amplification reaction mixture in a limiting amount relative to the other primer, which known as the Excess Primer. The amplification proceeds to generate both product strands exponentially, until the Limiting Primer is exhausted. The amplification reaction continues utilizing only the other primer (the Excess Primer), producing single-stranded amplification product, or “amplicon”. Non-symmetric PCR methods include asymmetric PCR, wherein the concentration of one primer of a symmetric PCR primer pair is reduced, generally by a factor of at least five, and LATE-PCR.

As used herein, “LATE-PCR” means a non-symmetric DNA amplification employing the PCR process utilizing one oligonucleotide primer (the “Excess Primer”) in at least five-fold excess with respect to the other primer (the “Limiting Primer”), which itself is utilized at low concentration, up to 200 nM, so as to be exhausted in roughly sufficient PCR cycles to produce detectable double-stranded amplicon, said cycle being known as the threshold cycle, C_{T} value, wherein the concentration-adjusted melting temperature of the Limiting Primer to its fully complementary sequence is equal to or higher than the concentration-adjusted melting temperature of the Excess Primer at the start of amplification, preferably at least as high and more preferably 3-10° C. higher; and wherein thermal cycling is continued for multiple cycles after exhaustion of the Limiting Primer to produce single-stranded product, namely, the extension product of the Excess Primer, sometimes referred to as the “Excess Primer Strand”. Amplification and detection assays utilizing non-symmetric PCR methods may utilize “low-temperature” probes, wherein the Excess Primer is in at least five-fold excess with respect to the Limiting Primer and the Tm of the detection probe is at least 5 degrees below the Tm of the Limiting Primer.

“Melting temperature” (Tm) is the temperature at which 50% of an oligonucleotide exists in double-stranded form, and 50% exists in single-stranded form. In LATE-PCR methods described this application Tm's of primers are concentration-adjusted values [Tm₀] calculated for complementary or mismatched nucleotide sequences using the software program Visual OMP (DNA Software, Ann Arbor, Mich.) which uses a proprietary modification of the “nearest neighbor” method (Santa Lucia, J. (1998), PNAS (USA) 95: 1460-1465; and Allawi, H. T. and Santa Lucia, J. (1997), Biochem. 36: 10581-10594), with salt concentrations typically set to 50 mM monovalent cation and 3 mM divalent cation. For reagent additives and for probes, Tm's are initially calculated by that method, ignoring covalently bound moieties and secondary structures. Because reagent additives and probes include covalently bound moieties such as fluorophores and quenchers, it is understood that the actual Tm's may differ slightly from calculated values due to actions of such moieties. If an amplification reaction mixture containing a reagent additive is subjected to a melt analysis (or an anneal analysis), an actual, or “observed” Tm of the reagent additive in that reaction mixture is obtained. Actual Tm's of labeled probes can generally be determined empirically, including structured probes such as molecular beacon probes.

“Selectivity” as used herein means the preference of a DNA polymerase to extend a recessed 3′ end of a hybrid when the 3′ terminal region, particularly when the terminal 3′ nucleotide, of the recessed 3′ end is perfectly complementary, that is, is hybridized with no mismatch. Stated another way, selectivity is selection against a 3′ terminal priming sequence that is not perfectly matched to its target. Selectivity against 3′ terminal-region mismatches applies to primer-target hybrids, which signifies the preference of a polymerase for a perfectly complementary hybrid over a primer-target hybrid having a mismatch at, for example, the 3′ terminal nucleotide. Improvement in discriminating against mismatched primers is sometimes referred to as improvement in “polymerase selectivity” or an improvement in “primer specificity.” More generally, selectivity against 3′ terminal-region mismatches also applies to hybrids having recessed, extendable 3′ ends formed by any two DNA strands in an amplification reaction mixture, for example, if one synthesized strand (an amplicon strand) hybridizes to (that is, primes on) another amplicon strand. The measure of selectivity in PCR and other thermal cycling amplification reactions is the difference (ΔC_{T}) between the threshold cycle (C_{T}) of the signal from amplification of the non-preferred hybrid, for example the hybrid formed by a primer and a mismatched target and the C_{T} of the signal from amplification of the preferred hybrid, for example the hybrid formed by a primer and a matched target. Improvement in selectivity due to a change in the reaction mixture, such as, for example, the use of a reagent additive, is the net C_{T} difference (ΔΔCT) obtained by subtracting the ΔC_{T} without the change (for example, without any reagent additive) from the ΔC_{T} that results with the additive. In other words, the value of the threshold cycle, C_{T}, of a perfectly match primer to its template strand is always smaller than the value of the C_{T} of a mis-matched primer to the template strand, and inclusion of an additive can increase the difference between these C_{T} values.

Increased polymerase selectivity is manifest in at least one of the following ways: 1) suppression of mis-priming; 2) increased primer specificity for perfectly complementary primer-target hybrids and against hybrids having recessed 3′ terminal sequences that are not perfectly complementary; 3) increased polymerase selectivity for primer-target hybrids having recessed 3′ terminal sequences that are GC-rich and against hybrids having recessed 3′terminal sequences that are AT-rich; 4) suppression of product evolution; 5) reduced scatter among replicate reactions.

References are made to polymerase efficiency. By “efficiency” is meant the rate of a polymerase activity, either the quantified kinetics of probe cleavage via primer-independent 5′exonuclease activity or the quantified kinetics of product amplification via polymerase activity. The kinetics of primer-independent probe cleavage is manifested as the slope of the curve of production of cleaved fragments. The effect of a change in an amplification reaction, for example, inclusion of a reagent additive, is evidenced by a change, generally a reduction, in slope. The kinetics of polymerase activity is manifested as the C_{T} of an amplification reaction. The effect of a reagent additive is evidenced by a change, generally an increase, in C_{T} between amplification of a perfectly matched target with the reagent additive and without the reagent additive. We sometimes refer to negative kinetic effects as “inhibition” Inhibition is also evidenced by a reduction in the production of amplified products, which may be shown by a reduction in intensity of SYBR or probe signal or by diminution in the magnitudes of peaks and valley in first-derivative curves.

“Type A Reagent” means a hairpin shaped single stranded oligonucleotides modified at both the 3′ and 5′ ends so that the end of the stem is stabilized relative to a DNA-DNA hybrid, such as by addition of dabcyl moieties, by addition of Black Hole Quencher™ moieties, or by inclusion of 2′ O-methyl nucleotides at the end of the stem. The stem-loop oligonucleotides have loops of 3-22 nucleotides and stems having calculated Tms of 50-85° C. Type A Reagents and assays utilizing them are described in our published international patent application WO 2006/044995.

“Type B Reagent” means a pair of complementary or partially complementary oligonucleotides that form a hybrid 6-50 nucleotides long, wherein the oligonucleotides are modified on one or both ends by addition of polycyclic moieties, for example, dabcyl or coumarin moieties, that do not have bulky portions that are non-planar. Type B Reagents and assays utilizing them are described in our published international patent application WO 2010/10507

“Type C Reagent” means a pair of oligonucleotide strands that form a hybrid at least six nucleotides long with a calculated Tm of at least 32° C., wherein strands in the terminal region of the hybrid the strands contain interacting label moieties, a fluorophore on one strand and either a fluorophore or a non-fluorescent quencher on the other strand. Type C Reagents and assays utilizing them are described in our co-pending U.S. provisional patent application No. 61/755,822, filed Jan. 23, 2013 titled “Reagents for Improving PCR Accuracy”.

“Type D Reagent means a relaxed (long), closed, circular double-stranded DNA.

“Type I DNA polymerase” (or “Type I polymerase) has its customary meaning in the art. Type I polymerases, sometimes referred to as Pol I, are implicated in DNA repair. They have 5′to3′ polymerase activity and, both 3′to5′ exonuclease activity (proofreading) and 5′to3′ exonuclease activity (RNA primer removal). Examples of Type I polymerases are Taq DNA polymerase, Tfi DNA polymerase, and pfu DNA polymerase. As used herein, “Type I polymerase” includes modified enzymes such as Tfi polymerase and pfu⁻ DNA polymerase.

### SUMMARY

This invention includes compositions that are or include Type I DNA polymerase molecule that is covalently modified for improved selectivity relative to unmodified polymerase. In such compositions a Type I DNA polymerase molecule is crosslinked to a molecule selected from the group consisting of Type A, Type B, Type C and Type D Reagents (collectively, Type A-D Reagents), or to another Type I DNA polymerase molecule. Compositions according to this invention include Type I polymerase that is dimerized, either directly or through a bridge molecule selected from the group consisting of Type A, Type B, Type C or Type D Reagent, and compositions containing the same. Type I DNA polymerases include, for example, Taq DNA polymerase, pfu DNA polymerase, Tfi+ polymerase, Tfi− polymerase, and other Type I Polymerases.

Preferred compositions are or include dimerized Type I polymerase containing two polymerase molecules, for example, two molecules of Taq DNA polymerase, covalently linked to each other either directly or through Type A, Type B, Type C or Type D Reagent, preferably a Type A-C Reagent, more preferably a Type B Reagent. Compositions according to this invention may include mixtures, for example, dimerized Taq covalently linked through such a Reagent plus monomeric Taq covalently linked to the Reagent plus unbound monomeric Taq.

This invention includes methods utilizing any of the foregoing modified polymerase compositions, for example, primer-dependent linear and exponential DNA amplifications such as a PCR amplification (including symmetric PCR, asymmetric PCR and LATE-PCR). Amplification methods according to this invention may include detection of amplified products either during amplification, such as with a dsDNA binding dye, for example SYBR Green, or with fluorescently or luminescently labeled hybridization probes, for example molecular beacon probes, or following amplification, such as, for example, by gel electrophoresis, end point detection with dye or probes, or post-amplification melt analysis.

This invention also includes reaction mixtures for performing any of the foregoing methods, where the reaction mixture contains one of the foregoing modified polymerase compositions.

This invention also includes reagent kits for performing any of the foregoing methods, where the kit contains, in addition to one of the foregoing modified polymerase compositions, suitable amplification reagents, for example, MgCl₂, salt and dNTPs, and where applicable, dsDNA binding dye, labeled hybridization probes, or both.

Preferred compositions are or include a stable dimeric Type I polymerase. The dimeric polymerase may be purified to remove monomeric polymerase, or the composition may additionally include monomeric polymerase, either crosslinked to a Type A-D Reagent or un-crosslinked, or both. Methods of this invention utilizing stable dimeric Type I polymerase achieve improved selectivity without requiring the presence of unbound Type A-D Reagent molecules. Depending on the specific crosslinked structure, preassembled homo-dimers of Taq polymerase, Tfi+ polymerase, Tfi− polymerase or other Type I polymerase undergo allosteric closure of all polymerase catalytic sites upon binding of one or more DNA templates in the presence of dNTP, or preassembled dimeric enzymes have a “closed”, more selective, polymerase catalytic sites prior to binding DNA.

Less preferred compositions are or include monomeric Type I polymerase crosslinked to a Type A-D Reagent. With such compositions, an amplification reaction mixture includes un-crosslinked Type I polymerase such that dimeric Type I polymerase forms in situ without the need for unbound Type A-D Reagent in the reaction mixture.

Type I polymerase crosslinked to (monomeric) or through (dimeric) a Type A-D Reagent can be made by preassembly of polymerase and Reagent followed by DNA/protein crossinking after a double-stranded DNA molecule plus dNTP has been bound to one at least one enzyme monomer. Type A, B, or C Reagents labeled with two, three or four chemical moieties, or unlabeled Type D Reagents can be used for preassembly. The results described in Example 3 show that it is also possible to assemble enzyme dimers using short oligomers of unlabeled double-stranded DNA. Assembly of enzyme dimers can be monitored by a variety of commonly used molecular biology techniques. Example 3, for instance, compares the migration of Taq polymerase alone in a non-denaturing gel with that of Taq polymerase mixed with either a double-stranded unlabeled oligomers that is 26 base pairs long, or with a Type B Reagent double-stranded DNA oligomer that is 22 base pairs long and is labeled with four dabcyl groups, one on each strand end. The results show that both the unlabeled DNA and the dabcyl-labeled DNA slow down the rate at which the protein migrates in the gel. Formation of the more slowly migrating band of protein occurs at the expense of the more rapidly migrating Taq-only band and is dependent on the concentration of DNA added to the protein. Both the protein and the DNA are negatively charged (i.e. they migrate in the same direction in the gel). These facts together indicate that the slower migrating DNA/Protein complex is due to a significant increase in the molecular weight of the complex, consistent with formation of dimeric Taq. As one skilled in the art will appreciate the amount of polymerase relative to the amount of the DNA ligand to generate either monomeric polymerase/ligand or dimeric polymerase/ligand can be established empirically. As one skilled in the art will also appreciate, the amount of the DNA ligand required to generate the highest proportion of enzymatically high specificity dimeric enzyme, rather than enzymatically inhibited dimers can be established empirically. We prefer Type B Reagent for preassembly and crosslinking. Optimization of particular preassembly and crosslinking methods generally includes setting variables such as inclusion or omission of dNTP, depletion or removal of dNTP present initially, omission or addition of Mg²⁺, inclusion of unlabeled DNA.

Crosslinking of polymerase to DNA ligand can be accomplished in any suitable manner, generally in a conventional manner. Reaction conditions required for crosslinking DNA and protein are well known, as are the chemical agents for DNA/protein crosslinking. These chemical agents include various aldehydes (including formaldehyde) and psoralen. As one versed in the art will appreciate the optimal agent, concentration, length and conditions for crosslinking the DNA and protein can readily be worked out empirically. Preassembled polymerase dimers are stable, even in the absence of DNA. Therefore preassembled dimers can be physically separated from monomers, for example, by size on gel filtration columns or by size in an electrophoretic field or gel. Another way to purify dimeric enzymes with crosslinked DNA is to temporarily bind the DNA/protein complex to a surface, for example, by an added biotin group and a streptavidin-coated bead or surface. The loop of Type A Reagents is also good for this purpose because it is single-stranded and lies outside of domains of the enzyme to which it is crosslinked. The loop can be hybridized to a complementary oligonucleotide on a bead, or the loop can have an incorporated biotin labeled nucleotide which can bind to a streptavidin-coated bead or surface.

The enzymatic properties of preassembled, purified Type I polymerase dimers can be assessed in terms of their capacities to carryout DNA synthesis, 5′exonuclease digestion and polymerase selectivity of perfectly matched versus mis-matched primers using amplification assays, including but not limited to the various assays described above and in prior patent applications for Type A, B, and C Reagents.

Crosslinking of proteins is also well known, and commercial strategies for protein crosslinking are available in the art. One useful approach is to use heterobifunctional crosslinking reagents. Perhaps the best known such reagent is SMCC. Yet another way to form stable, catalytically high specificity dimeric Taq is via modification of the protein itself. The chemical groups that stabilize protein/protein interactions are well known and include both non-covalent bonds and covalent bonds. Moreover, modification of proteins can change protein-protein interactions. Protein-Protein interactions can also be manipulated by methods of protein engineering. Protein-Protein interactions can be predicted on the basis of bioinformatics, as well as by direct experimental observation.

### BRIEF DESCRIPTION OF THE FIGURES

FIG. 1 is a schematic representation of the simple “Pac-Man” model of Type I polymerase activities.

FIG. 2 is a schematic representation of the interaction of Type I polymerase with increasing concentrations of Reagent.

FIG. 3 is a schematic representation of dimerized Type I polymerase interacting with a Reagent and with a primer to be extended.

FIG. 4 is a computer-generated representation of a Taq molecule.

FIG. 5 is an electrophoretic gel as described in Example 3.

FIG. 6 is an electrophoretic gel as described in Example 3.

FIG. 7 is an electrophoretic gel as described in Example 3,

### DETAILED DESCRIPTION

DNA is a right handed antiparallel double helix comprised of two oligonucleotides strands that run in opposite directions, that is, 3′-to-5′ and 5′-to-3′. When DNA is replicated, the two strands are temporarily unwound by a DNA topoisomerase and DNA helicase, which is part of the Type III DNA polymerase complex. The resulting single strands serve as templates for the synthesis of new base-pair complementary strands. Nucleotide precursors incorporated into new DNA strands during the process of replication are always incorporated in the 3′-to-5′ direction, because the process of replication is energized by cleavage of the 5′pyrophophates from the nucleotide triphosphate precursor upon addition of each new nucleotide. As a consequence of these facts, one new DNA strand, the leading strand, grows continuously into the replication fork via the action of DNA polymerase III, while the other strand, the lagging strand, is synthesized discontinuously. Discontinuous synthesis begins by synthesis of an RNA primer by the action of Primase. The RNA primer is then extended by incorporation of deoxynucleotides by the action of Polymerase III. Once the resulting Okazaki fragment abuts the 5′ end of the RNA primer, the primer is digested by the action of Type I DNA polymerase which also acts to extend the 3′ end of the Okazaki fragment. The resulting gap between the 3′ end of the fragment and the 5′ end of the lagging strand is closed by the action of DNA ligase. Taq polymerase and other thermal stable DNA polymerases used in PCR amplification are Type I polymerases. They have a 5′ to 3′ exonuclease activity in addition to the 3′ to 5′ polymerase activity.

Xray crystallographic studies of individual Type I Taq polymerase monomers show this protein to be a monomeric molecule comprised of three non-identical domains: the Polymerase Domain at one end, the 5′Exonuclease Domain at the other end, and the 3′Exonuclease Domain in between. The Polymerase Domain of the enzyme is described as a having the shape of a right-handed palm with fingers and a thumb. The double-stranded portion of a replicating DNA template lies across the palm, thereby placing the 3′end of the primer strand at the catalytic site. Binding of the DNA molecule causes the fingers and the catalytic site to move closer to the 3′ end where a new nucleotide is added. The extended 5′ template strand slips between the fingers and the thumb. Relative to the palm side of the polymerase, the catalytic site of the 5′exonuclease domain is located on the back side of the 5′ Exonuclease domain, the “wrist”. In addition, the 5′exonuclease domain of the enzyme is connected to the rest of the enzyme by a highly flexible polypeptide chain, which is thought to allow the 5′exonuclease domain to swivel.

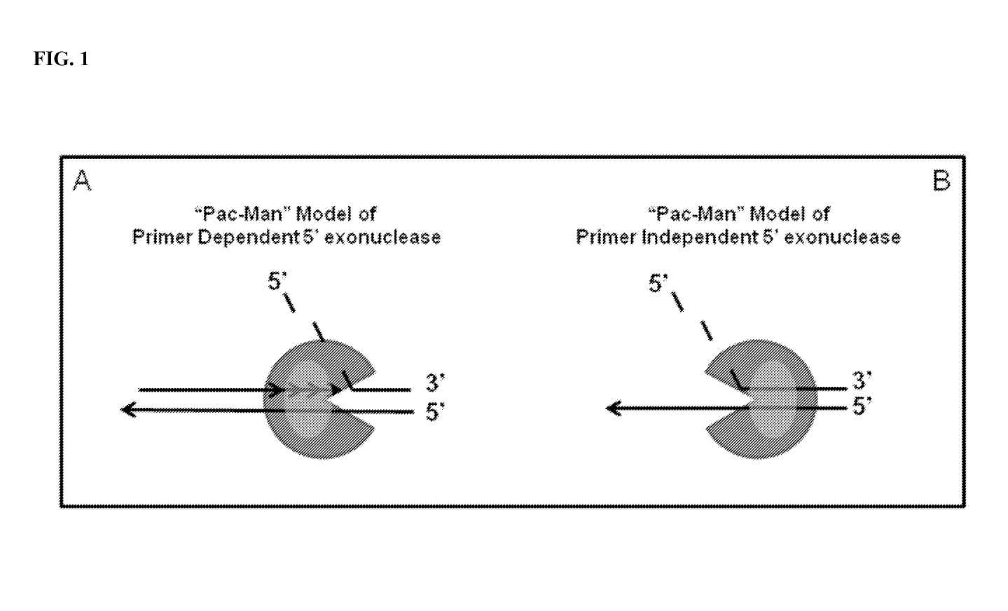

Type I polymerase enzymes are currently pictured as a monomeric enzyme, meaning that only one enzyme complex, one the lagging strand, is present. Taq polymerase in vitro exhibits both primer-dependent and primer-independent 5′ exonuclease activity. A simple “Pac-Man” model of these activities is routinely presented of primer-dependent 5′ exonuclease coupled with DNA synthesis by extension of the 3′end of a primer. See FIG. 1A). In this model the monomeric enzyme 1 is circular with a pie shaped “mouth” 2 for the 5′exonuclease, and the polymerase domain is located somewhere in the interior of the enzyme. As the model suggests the enzyme removes the 5′ end 3 of the strand 4 being repaired in its way by repeated “bites”, one nucleotide at a time. FIG. 1B extends this model to primer independent 5′exonuclease activity. In this case, the enzyme 1 approaches the overhanging 5′end 3 in the opposite direction, cleavage is not coupled to extension of the 3′ end of a primer. Primer-dependent cutting of a 5′end is known to require the 3′ OH on the nucleotide of the primer, but there is no such —OH group involved in primer independent cutting. Repeated cycles of primer independent cleavage require oscillation of the reaction temperature in the range of the Tm of the oligonucleotide to the template. In the absence of temperature oscillation the 5′exonuclease only trims the 5′ end once.

In vitro, particularly in a symmetric PCR amplification, the concentration of double-stranded template molecules and double-stranded amplicons is extremely low at the start of the reaction, gradually increases, and then becomes very high at the end of the reaction. This exponential change in double-stranded DNA (ds-DNA) concentration means that the enzyme will act as monomer initially with low primer specificity, then as an enzyme dimer with high specificity, and then the enzyme will be inhibited and the reaction will plateau. These are just the events observed in a typically symmetric PCR amplification. In the case of LATE-PCR or asymmetric PCR amplification, the inhibited dimer state will not be reached because the concentration of ds-DNA amplicons never becomes very high due to the relatively low concentration of the limiting primer. However, a typical LATE-PCR or asymmetric PCR amplification will still suffer initially from the low specificity of the monomeric form of the enzyme.

We have discovered that the Type 1 polymerase functions in a dimeric fashion, meaning that two molecules of enzyme interact in opposite orientations and thereby have the capacity to simultaneously bind both the leading strand and the lagging strand. While not wishing to be bound by any theory, we believe that Taq and related Type I polymerases can form a homo-dimeric structure such that the polymerase domain of one enzyme lies adjacent to the 5′exonuclease domain of a second polymerase molecule, and visa versa. The resulting dimer binds to the double strand emerging from the leading strand and to slide away from the replication fork, thereby allowing the other half of the dimeric molecule to digest the RNA primer and extend the lagging strand of the Okazaki fragment.

Binding of the dimer to the leading strand has an allosteric effect on both polymerase domains of the dimer, thereby improving the specificity of the polymerase synthesizing the lagging strand. Again while not wishing to be bound by any theory, this novel model for Type I polymerase function in a living cell might or might not be true for Taq polymerase in vitro due to the absence of the leading strand. However, addition of long double-stranded DNA molecules to an in vitro reaction overcomes this limitation. Addition of short DNA double stranded oligonucleotides at high concentration also enhances dimeric enzyme formation. Finally, addition of high concentrations of Reagents A-D at the start of an amplification reaction have been shown to inhibit amplification, which we believe to be due to binding in both polymerase domains of the homo-dimer.

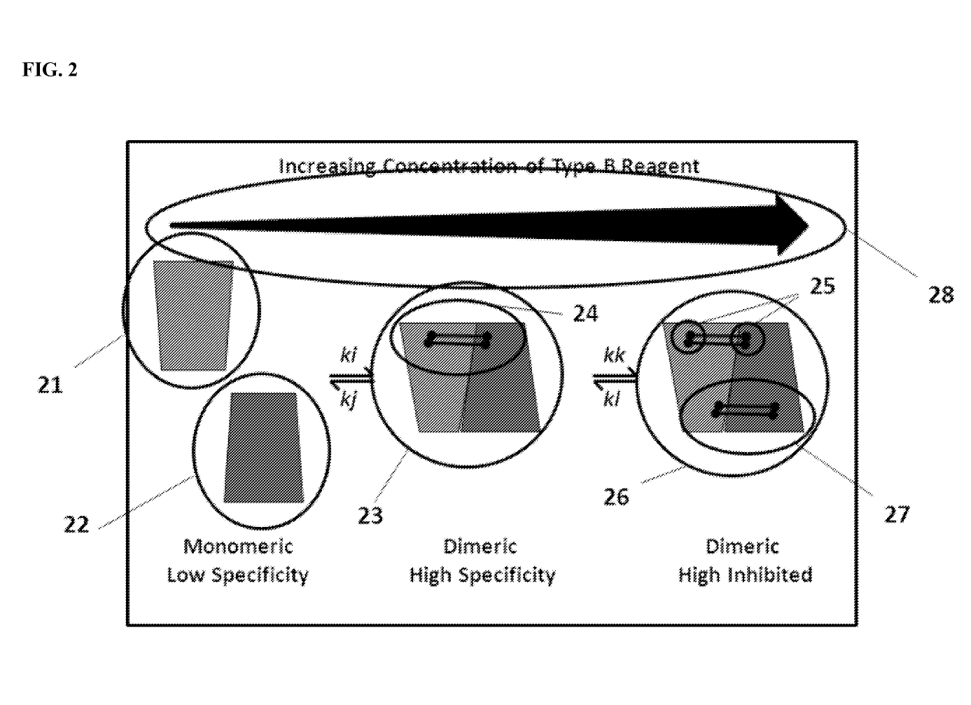

FIG. 2 illustrates dimerization utilizing one of the Reagents A-D. For this illustration we utilize a Type B Reagent 24 containing four terminal dabcyl groups 25. Starting with two monomeric Type I polymerase molecules 21, 22 and Type B Reagent molecules 24, there is shown what happens with the addition of increasing concentration of Type B Reagent, indicated by arrow 28. First a dimer 23 with the monomers oriented in opposite directions. If the concentration of Type B Reagent 24 is increased to a high level, dimer 26 with additional Type B Reagent molecule 27 is formed. The equilibrium between the monomeric state (21, 22) and dimer 23 can be described thermodynamically as the ratio of the equilibrium constant of formation (ki) to the equilibrium constant of disassociation (kj). That ratio varies with the structure and concentration of the particular Reagent B. The equilibrium between the dimer 23 and dimer 26 can be described thermodynamically as the ratio of the equilibrium constant of formation (kk) to the equilibrium constant of disassociation (kl). That ratio varies with the structure and concentration of the particular Reagent B. In general the ratio of ki to kj and the ratio of kk to kl both decrease in the following order of Reagent structures: long double-strand without modification, Type B, Type C, Type D, Type A.

We investigated the structure of Type I polymerase, using the embodiment of Taq, and Reagent, using the embodiment of a particular Type B Reagent, using a docking program, as reported below in Example 1. The results of the docking program GLIDE show that both the 5′ dabcyl G phosphate and the 3′ dabcyl C phosphate have reasonable binding GLIDE interaction scores for both the polymerase and 5′ exonuclease sites of Taq polymerase. Both a 3′ dabcyl C phosphate and a 5′ dabcyl G phosphate can dock into the polymerase site since both are able to fit into the double stranded site at the same time, thus completely blocking the site. This would occur when there is a very large concentration of Type B Reagent concentration, thus shutting down enzyme function. With the polymerase site filled as above, the 5′ exonuclease site may be filled with either a 3′ dabcyl C phosphate or a 5′ dabcyl G phosphate. Both fit well into the 5′ exo channel.

All of the docking results show that at certain Type B Reagent concentrations the 5′ exonuclease site will be blocked by either a 3′ dabcyl C phosphate or a 5′ dabcyl G phosphate, which indicates that Type B Reagent at even low concentrations will affect the 5′ exonuclease site, and therefore reduce the ability of Taq to create pseudo primers from 5′ DNA strands that have been cleaved by the 5′ exonuclease activity.

While not wishing to be bound by any theory, our computer modeling indicates how Type A-D Reagents assemble dimeric Type I polymerase. Allosteric effects involve a change in protein shape upon binding of a substrate. Taq polymerase, for example, is known to undergo an allosteric effect when double-stranded DNA binds to the palm of the enzyme in the presence of dNTP. In the absence of bound DNA, or the absence of dNTP's the fingers and thumb form a wide cleft in the enzyme, When DNA binds to the palm in the presence of dNTP the fingers and thumb come together and hold the 3′ end of the DNA strand tightly in the active site of the polymerase.

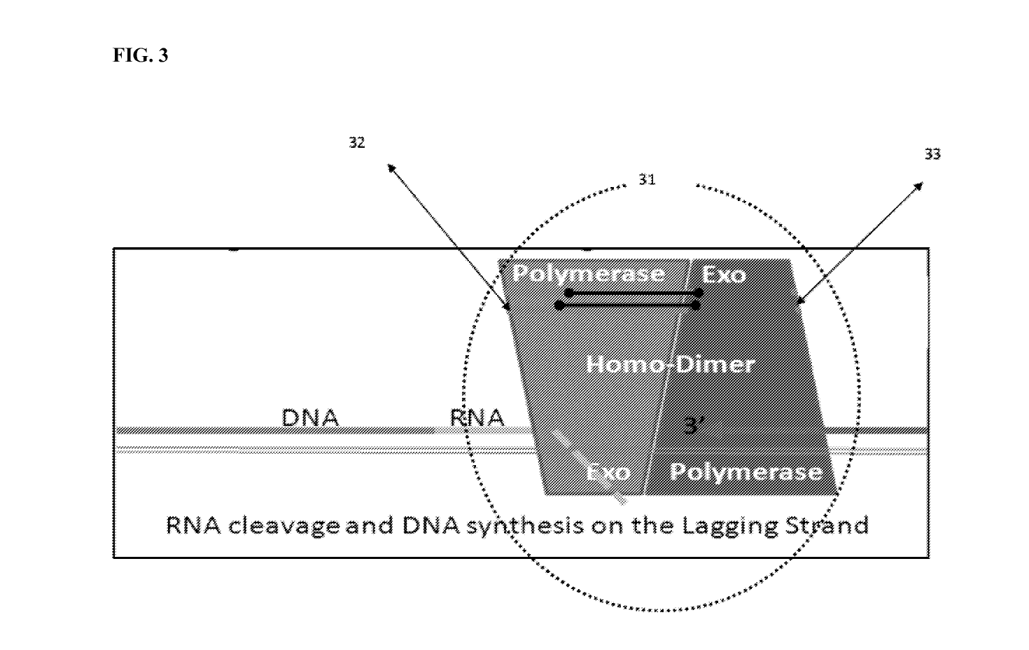

This allosteric effect is incompatible with the fact that Type A, B, and C Reagents at appropriate concentrations all increase polymerase selectivity of a matched versus a mis-matched 3′ end of a primer, if the enzyme functions as monomer. But this allosteric effect is compatible, indeed plausibly accounts for, how a single Type A, B, or C Reagent bound to a Taq dimer (dimer 23 in FIG. 2) increases the polymerase selectivity of the complex. The reason for this conclusion is this. If the Taq functions as a monomer and binds a Reagent, that reagent must sit in the palm of the enzyme where it might, indeed, cause the polymerase binding site to close. However, the Reagent would thereby be a competitive inhibitor to a primer/template hybrid. The only way for the enzyme to bind to the primer/template complex would be to release the Reagent, but this would require the fingers and thumb to open again, thereby eliminating any increase in selectivity accrued from binding the Reagent. In contrast, in the dimeric model the Reagent can bind to the palm of one enzyme molecule where it would be held by the closing of the fingers and thumb. This is shown schematically in FIG. 3, which depicts Type I polymerase dimer 31, comprised of oppositely oriented monomers 32, 33. The bottom polymerase site is blocked by Type B Reagent molecule 34, but the other polymerase site is active on strand 35. Importantly, this allosteric change in the shape of the dimer could include a change in shape of the opening between the fingers and the thumb of the second enzyme molecule, making it less receptive to a mis-matched primer/template hybrid.

Example 1 shows the results of both 3′ dabcyl C phosphate and 5′ dabcyl G phosphate binding to both polymerase site and to the 5′exonuclease site of Taq polymerase. These are the dabcylated ends of a Type B Reagent molecule that served as a predictor of Type B Reagent-Taq interactions. The orientation of the dabcyl moieties necessitates that the attached double-helix of the Type B Reagent is positioned outside of the Taq Polymerase with no contact with the enzyme's “palm” which is known to bind double-stranded DNA. This observation poses a major conundrum.

In order to resolve this conundrum we assessed how these models reflect upon other experimental evidence as well as additional information in the literature about Taq polymerase function. These data indicate that a far more plausible model for Taq/Reagent interactions involves binding of the double-stranded DNA of the Reagent to the “palm” of the enzyme adjacent to the active site of the polymerase domain. However, in this position the dabcyl moieties of the Reagent would not be able to bind the 5′ exonuclease site located on the back side of “wrist” in the same enzyme monomer.

Construction of a Taq dimer provides a solution to the conundrums posed by the combined results of Example 1 and reported experimental evidence. Our solution is the discovery that two Type I (for example Taq) polymerase molecules actually function as a dimer. Using computer modeling of dimer molecules reported in Example 2, we found that in all of the dimer models, the 5′ exonuclease site of one Taq monomer is positioned close to the polymerase site of the second Taq monomer. The two sites are now close and no longer 80 Angstroms apart as in a single Taq molecule. In the case of Type B Reagents, if the double-stranded DNA of the Reagent is bound to the “palm” of one monomer, either the 3′ or 5′ dabcyls of the Type B Reagent can bind to the 5′ exonuclease site on the back side of a second enzyme monomer. These models allow the catalytic site of the polymerase on one polymerase monomer to function in concert with the 5′exonuclease catalytic site on a second polymerase monomer. Coordinated function of these two enzymatic activities is required for the efficient continuous extension of the 3′end of an elongating strand confronted with the 5′ end of a strand bound to the same template.

Based on these findings, we conclude that in the presence of a single Type B Reagent molecule, a Taq polymerase dimer is formed that has the double-stranded portion of the Type B Reagent molecule located in the “palm” of one enzyme monomers, as described above, and either the 5′ or 3′ dabcyl in the 5′exonuclease channel on the second monomer, consistent with the GLIDE score docking data of Example 1. The binding of the Reagent is very likely to trigger an allosteric change in the shape polymerase site in both monomers. This result also explains how Type B Reagents function throughout PCR amplification to enhance polymerase selectivity at the unoccupied polymerase “palm” and its active site. Moreover, because two molecules of Taq function in concert in the dimer, the 5′ exonuclease site of one monomer is precisely positioned close to the polymerase site of the other monomer responsible for primer extension. Finally, when moderate to high levels of Type B Reagent are present in the reaction, the unoccupied “palm” of the second monomer selectively binds a matched primer on its template strand and proceeds to extend that primer. Under these cirumstances the 3′ end of the match primer is extended nucleotide by nucleotide via the correct complementary base pairing of the next nucleotide to be covalently linked to the 3′ end of the extending primer. Under in vitro conditions the dimer is stabilized during these steps by the bound Reagent. It is likely that under in the living bacterium the dimer is stabilized by the already completed double-stranded DNA of the leading strand.

We have further considered whether the dimer model of Taq polymerase sheds light on the functionality of Taq polymerase in vivo. The dilemma in the understanding of the functioning of Taq polymerase during extension of the 3′end of the primer is how the 5′ exo site works with the polymerization site to add nucleotides when there is a 5′ overhang that must be excised before a nucleotide is added. The first base of the overhang must be cut as another base is added directly before it along the primer strand. This means that the polymerase site and the 5′ exo site must be remarkably close to each other and not 80 Angstroms (80 A) away as is shown in the crystal structure. Allowing two Taq molecules to come together and form a dimer allows the 5′exo site to lie very close to the polymerase site as shown by computer modeling.

Evidence for dimerization is provided by electrophoretic gel separation studies reported in Example 3. The results presented in Example 3 indicate a higher order oligomeric state for Taq. The native gels showed a series of Taq bands that migrate to approximately the same position in each gel in various ratios of the EP020 and unlabeled double-stranded DNA. The appearance of only a single band in a gel lane containing only Taq shows that the Taq migrates to a single place in the lane. The appearance of only a single band in the ratio of 0.5:1 (unlabeled double-stranded DNA:Taq) means that all of the Taq is part of the species forming this band. The ratio indicates that the band is a dimer. Further studies will be needed to confirm the dimerization, and examine the structure of the species responsible for the band. As stated previously it is believed that X-ray crystallography will be key to understanding the binding behavior of Taq.

FIG. 2 illustrates how increasing concentrations of double-stranded DNA leads to the formation and stabilization of dimeric Taq, with increase polymerase selectivity, with further increase leading to inhibition of polymerase activity. The balance between monomeric, dimeric, and inhibited dimeric conformations of the enzyme will depend on the concentration of the reagent, the affinity Taq for the reagent, and the rate at which the bound reagent dissociates from Taq. These variables, in turn, will be influenced by the number and types of modifying groups on the 3′ and 5′ ends of a Reagent. If the 3′ ends are simply capped with carbons to prevent elongation and the 5′end are not modified the molecule will be in the most “natural” state and can be expected to be released by Taq at the highest rate. Under these circumstances the proportion of monomers, dimers, and inhibited-dimers will simply depend on the concentration of the double-stranded oligonucleotide. In contrast, if 1, 2, 3, or 4 modifying groups with high affinity for Taq polymerase are added to the oligonucleotide, the “effective” concentration of the added reagent will increase, and the balance of monomer, dimer, and inhibited-dimer will shift toward the inhibited-dimer (as indicated by the large arrow 28 in FIG. 2). International patent application WO2010/105074 describes that Type B Reagents in the presence of a substrate and dNTP's can inhibit the 5′exonuclease activity at relatively low concentration and inhibit the polymerase activity at somewhat higher concentration. We believe that the added reagent preferentially forms a dimer with one bound Reagent and thereby alters the selectivity of other polymerase site via an allosteric change in the shape of the complex, but the same reagent at a higher concentration becomes a competitive inhibitor for the other polymerase site of the dimeric complex.

With Type C Reagents (double-stranded oligonucleotides labeled with bulky groups which do not inhibit the 5′exonuclease activity at low concentration and only marginally inhibit the polymerase activity at high concentration) the bulky groups keep the labeled end of the Reagent from entering into either binding site of the enzyme, but permit the unlabeled end to contact the enzyme. We believe Type C Reagent is easily released from the enzyme complex more rapidly than a Type B Reagent, but more slowly than an unlabeled double-stranded DNA molecule.

Type A Reagents inhibit both the 5′exonuclease activity and the polymerase activity of the enzyme at lower concentrations than do Type B and C Reagents. We believe that this is because the loop of the Type A Reagent fails to enter the binding pocket of the enzyme but the two dabcyls on the other end of the molecule readily bind to the enzyme.

Dimeric Type I polymerases can be formed by addition of relaxed closed circular double-stranded DNA, Type D Reagents. Type D Reagent, like Type B Reagent is likely to favor formation of enzyme dimers, because the double-stranded circle has no ends, and therefore the enzyme cannot slide off readily. Moreover, because each Type D Reagent is a long double-stranded DNA compared to Reagents Types A-C, it is likely that each closed circle binds more than one monomer of polymerase, for example Taq, and, hence, assembles more that one dimeric Taq complex. But, high levels of Type D Reagent are less likely than Type B Reagent to promote formation inhibited-dimeric Taq, because steric forces between closed circular DNA molecules keep them from coming close together. These properties of Type D molecules therefore favor the accumulation of high-specificity dimeric Taq. Currently, Type D Reagents are our less-preferred choice to achieve polymerase selectivity in PCR amplification reactions for several reasons. First, these closed circular DNA molecules have to be relaxed, or at least substantially relaxed rather than highly supercoiled, which is relatively difficult to achieve. Second, closed circular molecules are easily nicked, and nicked molecules with 3′ ends will replicate via a rolling circle type amplification kinetics.

Formation and dissociation of dimeric and monomeric enzyme complexes, as described above, are the result of chemical equilibria that depend on the presence of unbound (free) Reagent molecules in solution. In contrast, stable dimeric Type I polymerases do not require the presence of unbound Reagent molecules, because these molecules are either crosslinked to the enzyme or are no longer required for dimerization of crosslinked proteins. Preassembled homo-dimers of Taq polymerase, Tfi+ polymerase, Tfi− polymerase, and other Type I Polymerases are useful and valuable when these enzymes are prepared, stored, and used readily and inexpensively. Preassembled dimeric enzymes also exhibit increased polymerase specificity like polymerase prepared in PCR mixtures containing Type A-D Reagents. Based on our current understanding of mechanism, this means that preassembled dimeric enzymes would either still undergo allosteric closure of all polymerase catalytic sites upon binding of one or more DNA templates in the presence of dNTP, or preassembled dimeric enzymes would have a “closed”, more selective, polymerase catalytic sites prior to binding DNA.

Preassembled dimeric polymerases can be constructed and permanently stabilized by DNA/Protein crosslinking after a double-stranded DNA molecule plus dNTP has been bound to one at least one enzyme monomer. Type A, B, or C Reagents labeled with two, three or four chemical moieties, or unlabeled Type D Reagents can be used to assemble enzyme dimers prior to crosslinking. The results described in Example 3 suggest that it is also possible to assemble enzyme dimers using short oligomers of unlabeled double-stranded DNA. Assembly of enzyme dimers can be monitored by a variety of commonly used molecular biology techniques. Example 3, for instance, compares the migration of Taq polymerase alone in a non-denaturing gel with that of Taq polymerase mixed with either a double-stranded unlabeled oligomers that is 26 base pairs long, or with a Type B Reagent double-stranded DNA oligomer that is 22 base pairs long and is labeled with four dabcyl groups, one on each strand end. The results show that both the unlabeled DNA and the dabcyl-labeled DNA slow down the rate at which the protein migrates in the gel. Formation of the more slowly migrating band of protein occurs at the expense of the more rapidly migrating Taq-only band and is dependent on the concentration of DNA added to the protein. Both the protein and the DNA are negatively charged (i.e. they migrate in the same direction in the gel). These facts together show that the slower migrating DNA/Protein complex is due to a significant increase in the molecular weight of the complex, consistent with formation of dimeric Taq.

As one skilled in the art will appreciate, the amount of the DNA ligand required to generate the highest proportion of enzymatically high-specificity dimeric enzyme, rather than enzymatically inhibited dimers can be established empirically. And, as a person versed in the art will appreciate, additional variables can be varied to define the optimal conditions for construction of dimeric enzymes, including: omission of dNTP, depletion or removal of dNTP present initially, omission or addition of Mg²⁺, inclusion of unlabeled DNA. While any one of Reagents A-D can be used, it is preferred that Type B Reagent having either two labeled terminal nucleotides on one end be used, more preferred that Type B Reagent with three labeled terminal nucleotides be used, and most preferred that Type B Reagent with four labeled terminal nucleotides be used to assemble enzyme dimers prior to crosslinking. A Type B Reagent with two labeled terminal nucleotides, plus at least one biotin residue linked to one of the nucleotides on the other end of double-stranded oligonucleotide can also be used to assembly dimeric enzyme prior to crosslinking.

The reaction conditions required for crosslinking DNA and protein are well known, as are the chemical agents for DNA/protein crosslinking. These chemical agents include various aldehydes (including formaldehyde) and psoralen (see for instance, http://en.wikipedia.org/wiki/Crosslinking_of_DNA). As one versed in the art will appreciate the optimal agent, concentration, length and conditions for crosslinking the DNA and protein can readily be worked out empirically. Preassembled dimers will be stable, even in the absence of DNA. Therefore reassembled dimers can be physically separated from monomers. One way to separate monomers and dimers is by size on gel filtration columns or by size in an electrophoretic field or gel. Another way to purify dimeric enzymes with crosslinked DNA is to temporarily bind the DNA/protein complex to a surface. For instance, an added biotin group (see above) can subsequent be used to purify the protein enzyme complex, via binding to a streptavidin bead or surface. The loop of Type A Reagents is also good for this purpose because it is single-stranded and lies outside of domains of the enzyme to which it is crosslinked. The loop can be hybridized to a complementary oligonucleotide on a bead, or the loop can have an incorporated biotin labeled nucleotide which can bind to a streptavidin coated bead or surface.

The enzymatic properties of preassembled, purified Type I polymerase dimers can be assessed in terms of their capacities to carryout DNA synthesis, 5′exonuclease digestion and polymerase selectivity of perfectly matched versus mis-matched primers using the various assays described above and in prior patents and patent applications, cited above, for Type A, B, and C Reagents.

Yet another way to form stable, catalytically high specificity dimeric Taq is via modification of the protein itself. The chemical groups that stabilize protein/protein interactions are well known and include both non-covalent bonds and covalent bond (see for instance: http://en.wikipedia.org/wiki/Protein_dimer; http://en.wikipedia.org/wiki/Dimer_%28chemistry%29. Moreover, “modification of proteins can itself change protein-protein interactions. For example, some proteins with SH2 domains only bind to other proteins when they are phosphorylated on the amino acid tyrosine while bromodomains specifically recognize acetylated lysines” (http://en.wikipedia.org/wiki/Protein%E2%80%93protein_interaction). Protein-Protein interactions can be predicted on the basis of bioinformatics, as well as by direct experimental observation (see for instance, http://en.wikipedia.org/wiki/Protein%E2%80%93protein_interaction_prediction).

Protein-Protein interactions can also be manipulated by methods of protein engineering (see for instance, http://en.wikipedia.org/wiki/Protein_engineering & http://en.wikipedia.org/wiki/Enzyme_engineering). Crosslinking of proteins is also well known. Commercial strategies for protein crosslinking are presented at: http://proteinmods.com/applications/protein-crosslinking/?gclid=CKaW96mQ-rcCFXRo7AodxkAAdg. This company's website states, “Protein crosslinking is a useful technique to confirm protein-protein interactions, to generate protein molecules with bi-functionality, or to fix proteins at a desired location. One useful approach is to use heterobifunctional crosslinking reagents. Perhaps the best known such reagent is SMCC. SMCC reacts with primary amines on one end (via NHS-ester) and thiols on the other (via maleimide). Most proteins contain many primary amines but very few (if any) available thiols. One common crosslinking protocol would be to first react SMCC with a protein, and then remove unreacted SMCC. The SMCC activated protein is then exposed to a protein that has available thiols (sulfhydryls), allowing the formation of a covalent bond between the proteins.”

As a person skilled in the art will appreciate, additional variables can be modified to define the optimal conditions for construction of dimeric enzymes, including: omission of dNTP, depletion or removal of dNTP present initially, omission or addition of Mg²⁺. In addition, alterations of the amino acid composition of proteins can be changed to favor enzyme-to-enzyme interactions, as well as to enhance closure of the polymerase binding pocket. The principle of constructing an obligate protein dimer from a protein monomer has been described by Kuhlman et al. (“Conversion of monomeric protein L to an obligate dimer by computational protein design,” Proceedings of the National Academy of Science, USA. 98, 10687-10691, Sep. 11, 2001).

Compositions according to this invention include Type I polymerase molecules that are crosslinked either to other Type I polymerase molecules or to Type A-D Reagent. As one skilled in the art will appreciate, preassembly as described above followed by DNA-protein crosslinking may lead to a composition that includes multiple polymerase species, at least one of which is crosslinked. Considering preassembly as described in conjunction with FIG. 2, a composition according to this invention may include both dimeric polymerase species shown, that is, dimeric polymerase molecules both crosslinked to a single Reagent molecule (species 23), and dimeric polymerase molecules crosslinked to two Reagent molecules (species 25), the relative proportions being controlled. Such compositions may include as well unreacted monomeric polymerase molecules (21, 22 in FIG. 2), monomeric polymerase molecules reacted with single Reagent molecules, and possibly, though not likely, monomeric polymerase molecules reacted with two Reagent molecules. Similarly, preassembly as described above followed by protein-protein crosslinking, a composition according to this invention may include dimeric polymerase wherein the two polymerase molecules are crosslinked to one another, plus unreacted monomeric polymerase plus monomeric polymerase bound to crosslinking reagent. Following either type of crosslinking, unreacted Reagent may be removed. Further, a composition may be purified so as to contain only a single crosslinked species.

Characterization of compositions according to this invention may be accomplished by any appropriate method. As described in Example 3, monomeric and dimeric polymerase can be detected, for example, by electrophoretic separation in a non-denaturing gel. Differentiation of unreacted species, monomeric or dimeric, from crosslinked species can be accomplished by electrophoretic separation in a denaturing gel, such as a gel containing sodium dodecyl sulfate (SDS). Dimeric Taq with one EP020 can be distinguished from dimeric Taq with two EP020's by a functional test to determine whether a PCR amplification with the composition is shut down.

The various species of molecules described herein can be separated from each other using physical and chemical methods well known in protein biochemistry. Protein characteristics used for separation and purification include one or more of the following: molecular weight, surface charge, the presence of covalently bound ligands, the presence or absence of specific epitopes on the surfaces a protein, protein shape, protein solubility or insolubility in aqueous and non-aqueous solvents. Monomeric Type I polymerases can be separated from covalently crosslinked dimeric enzymes on the basis of molecular weight in columns, or gels, or electrophoretic fields. Monomeric enzymes can also be separated from dimeric enzymes on the basis of antibody binding to an epitope that is accessible on one protein but shield on the other. Monomeric and dimeric Type I polymerase(s) with a crosslinked nucleic acid reagent can be distinguished from monomeric and dimeric molecules to which the nucleic acid is non-covalently bound by radioactively labeling the nucleic acid with P³²-phosphate and testing whether the radiolabel migrates with the protein, or separately from the protein during electrophoresis in a denaturing gel, such as an SDS gel. Type A Reagents are particularly useful for separating and purifying the proteins to which they are covalently bound because one or more nucleotides in the single-stranded DNA in the loop of the Reagent can by labeled with a side-group which enables the DNA-protein complex to be retarded when it is passed over a column, or through a filter, or across a surface that temporarily binds the side-group.

Once the monomeric enzyme with a bound Reagent is separated from unbound Reagent, the protein-with-its ligand can be mixed with additional enzyme monomers to make a dimeric enzyme, and the specificity of these complexes can be established as described elsewhere herein. Similarly, specificity assays can be used to characterize crosslinked dimeric 5 proteins with and without a bound Reagent.

### EXAMPLES

### General

In modeling studies presented in the Examples that follow, we used Taq polymerase and a particular Type B Reagent, which we refer to as EP020. EP020 has four dabcyl moieties are attached to a double-stranded oligonucleotide of 22 bases in length. The sequence is as follows:

(SEQ ID NO.: 1)
5′ Dabcyl-GAAATAAAATAAAAATAAAATA-Dabcyl 3′
(SEQ ID NO.: 2)
3′ Dabcyl-CTTTATTTTATTTTTATTTTAT-Dabcyl 5′

### Example 1

### Molecular Modeling of Dabcyl/Taq Interactions

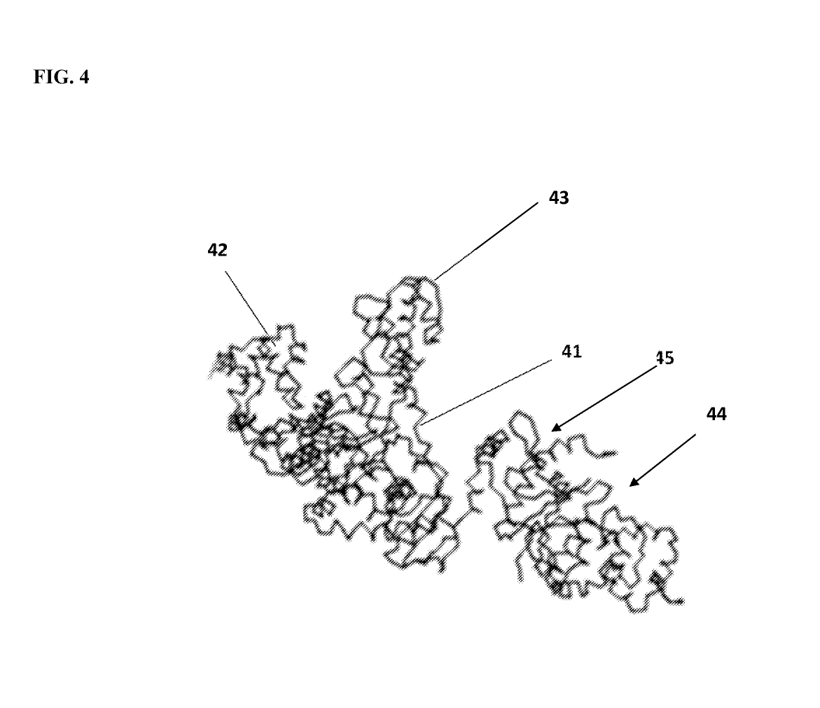

The question is how does EP020, a representative Type B Reagent, interact with Taq Polymerase? The crystal structure of monomeric Taq polymerase with and without a bound double-stranded DNA template in the polymerization site has been solved. FIG. 4 illustrates the resulting structure without bound DNA. Polymerase active site 41 lies between fingers 42 and thumb 43. The 5′ exonuclease active site 44 lies on the back side of wrist 45. In this Example we calculated the probability of 3′ and 5′ Dabcyl binding to the polymerase active site between the “fingers” and the “thumb” and the 5′ exonuclease active site on the back side of the “wrist”. Each region was analyzed separately, because the computational analysis is otherwise too large for our computers.

### Method

X-ray crystallography is the optimal method to achieve a complete understanding of Taq/Reagent interactions at the atomic level, but crystallographic studies are not yet complete. In the meantime we used docking program software for a small molecule plus a portion of a protein to calculate the probability of the end nucleotide phosphate and either a 3′ or 5′ dabcyl moiety interactions with either the polymerase site or the 5′ exonuclease site of Taq Polymerase.

The protocol for generating a GLIDE score is a program suite from Schrodinger: a user interface called Maestro, and a specific program used for docking is GLIDE (Grid-based Ligand Docking with Energetics. Glide Score (G Score) is given by:

G Score=a*vdW+Coul+Lipo+Hbond=Metal+BuryP+RotB+Site,
where
vdW=van der Waals energy
Coul=Coulomb energy
Lipo=Lipophilic contact term
HBond=Hydrogen-bonding term
Metal=Metal-binding term
BuryP=Penalty for buried polar groups
RotB=Penalty for freezing rotatable bonds
Site=Polar interactions in the active site′
and the coefficients of vdW and Coul are:
   a=0.065, b=0.130 fpr Standard Precision (SP) Glide

The GLIDE software essentially looks at all of the interactions between a docking molecule (a dabcyl) and a portion of a protein molecule (Taq polymerase), and computes the best interactions that have the lowest interaction energy. Interactions of the protein and the small molecule are defined in a three dimensional docking space, and the program computes a GLIDE score based on these interactions. The more negative the GLIDE score the better the interactions between the Taq and the molecule. Unfortunately, in the program GLIDE, the size of the small molecule is limited. Therefore we restricted our analysis to either the 3′ or the 5′ nucleotide phosphate linked to the corresponding dabcyl moiety. The four GLIDE scores for the best interactions were generated: 3′ dabcyl C phosphate at polymerase active site 41, 5′ dabcyl G phosphate at polymerase active site 41, 3′ dabcyl C phosphate at 5′ exonuclease active site 44, and 5′ dabcyl G phosphate at 5′ exonuclease active site 44.

### Results

### 3′ Dabcyl C Phosphate at Polymerase Site

The GLIDE program showed the 3′ dabcyl C phosphate docked into the polymerase site of Taq had a docking score of −8.08, which is a tight binding interaction score. The 3′ dabcyl sticks into the site while the C phosphate remains near the entrance to the site. It is also closer to the thumb part of the polymerase.

### 5′ Dabcyl G Phosphate at Polymerase Site

The GLIDE program showed the 5′ dabcyl G phosphate docked into the polymerase site of Taq had a docking score of −4.84, which is a moderate binding interaction score. The 5′ dabcyl sticks into the site while the G phosphate remains near the entrance to the site. It is closer to the fingers part of the polymerase

### 3′ Dabcyl C Phosphate at 5′Exonuclease Site

The GLIDE program showed the 3′ dabcyl C phosphate docked into the 5′ exo site of Taq had a docking score of −10.03, which is a tight binding interaction score. The 3′ dabcyl sticks into the site while the C phosphate remains near the entrance to the site. The molecule fits within the single strand channel, which lies on the underside of the 5′ exo part of the polymerase.

### 5′ Dabcyl G Phosphate at 5′ Exonuclease Site

The GLIDE program showed the 5′ dabcyl G phosphate docked into the 5′ exo site of Taq had a docking score of −5.33, which is a moderate binding interaction score. The 5′ dabcyl sticks into the site while the G phosphate remains near the entrance to the site. The molecule fits within the single strand channel, which lies on the underside of the 5′ exo part of the polymerase.

### Example 2

### Binding of a Type B Reagent to a Taq Dimer

Construction of a Taq dimer provides a solution to the conundrums posed by the combined results of Example 1 and reported experimental evidence. Our solution is the discovery that two Type I (for example Taq) polymerase molecules actually function as a dimer We postulate several possible configurations for dimer formation with a Reagent, which for ease of understanding we will describe with Taq as the polymerase and EP020 as the Type B Reagent. Configuration for two Taq polymerase molecules dimerized with one EP020 molecule is double-stranded DNA sitting in a polymerase channel and the 5′ dabcyl G phosphate end of EP020 sitting in a 5′ exonuclease site single strand channel. Two configurations for two Taq polymerase molecules dimerized with two EP020 molecules are double-stranded DNA sitting in a polymerase channel and a 5′ Dabcyl G phosphate end of EP020 sitting in the 5′ exonuclease sites. In the configuration with one EP020 molecule and in one configuration with two EP020 molecules, the 5′exonuclease site on one monomer is positioned close to the polymerase site and “palm” of the second monomer. In the other configuration with two EP020 molecules, there is only one closely apposed exo/pol site.

In all of these dimer models, the 5′ exonuclease site of one Taq monomer is positioned close to the polymerase site of the second Taq monomer. The two sites are now close and no longer 80 Angstroms apart as in a single Taq molecule. In the case of Type B Reagents, if the double-stranded DNA of the Reagent is bound to the “palm” of one monomer, either the 3′ or 5′ dabcyls of the Type B Reagent can bind to the 5′ exonuclease site on the back side of a second enzyme monomer. These models allow the catalytic site of the polymerase on one polymerase monomer to function in concert with the 5′exonuclease catalytic site on a second polymerase monomer. Coordinated function of these two enzymatic activities is required for the efficient continuous extension of the 3′end of an elongating strand confronted with the 5′ end of a strand bound to the same template.

### Example 3

### Native Gel Assays of Taq in the Presence of dsDNA

### Background

The computational models in Examples 1 and 2 show it is possible for Taq to form homodimers. Experimental evidence in support dimerization of Taq was obtained show using a traditional biochemical method, protein electrophoretic migration in a native (non-denaturing) gel. This approach is provides a relatively easily conducted, and repeatable, assay of the multimeric state of a protein in the presence/absence of a ligand.

Accordingly, we analyzed the native state of the Taq polymerase in the presence of various ratios of EP020 or a double stranded DNA oligomer. These tests were conducted to initially investigate the multimeric state of Taq.

### Methods

A sodium dodecyl sulfate (denaturing) polyacrylamide gel was first used to assess the quality of the Taq used in this Example. The gel was a 12% acrylamide gel (Sigma) and was run at approximately 200 V for 1 hour and 30 minutes. The molecular weight marker was the pre-stained Broad Range Marker (New England Biosciences).

The Taq for these experiments was prepared in a similar manner to the protocol given by Engelke et al. (“Purification of Thermus aquaticus DNA Polymerase Expressed in Escherichia coli,” Analytical Biochemistry, USA. 191, 396-400. December 1990). However, the protocol was modified to include elution of the protein into a buffer that was previously used for the crystallization of the Taq Kim, Y., et al. (“Crystal Structure of Thermus aquaticus DNA polymerase. Nature, UK. 376, 612-616. 17 Aug. 1995). The protein was kept at a stock concentration of approximately 10 mg/ml (100 μM). An SDS-PAGE gel was run to establish the purity of the Taq. Results (not shown) indicated that the sample contained the Taq and various lower molecular weight contaminants. The contaminants could have been a result of degradation of the Taq or could have been present in purification. The contaminant bands were also present in gels containing DNA and Reagent. Thus, they are not degradation products of Taq formed during incubation with the annealed dsDNA oligomers or Type B Reagent EP020.

The dsDNA oligomers for unlabeled double-stranded DNA were ordered from Eurofins MWG Operon. The two complementary strands were 26 nucleotides long and had the following sequences:

(SEQ ID NO.: 3)
Strand 1 = 5′-GCTGATCAAAAAACGGAATTGGACCC-3′
(blocked)
(SEQ ID NO.: 4)
Strand 2 = 5′-GGGTCCAATTCCGTTTTTTGATCAGC-′3
(blocked)

The calculated melting temperature of the two strands is approximately 70° C. The two oligomers were mixed, heated to 95° C. for approximately 5 minutes and then allowed to anneal at 55° C. for approximately 3 minutes. EPO20 sequences are given above. The calculated melting temperature of the two strands is approximately 55° C.

The blocked dsDNA or EP020 was then combined with the Taq in the ratios noted below. These mixtures were allowed to incubate at room temperature for 1 hour. Then the sample was placed through the electrophoretic separation to separate protein complexes in a native state. A non-denaturing polyacrylamide gel was used to separate the protein components of a solution by a charge to mass ratio. In this gel the charge on the protein is influenced by the pH of the buffer only. Assays were conducted using the protocol from Gallagher, S. (“One-Dimensional Electrophoresis Using Nondenaturing Conditions,” Current Protocols in Molecular Biology, 47, 10.2B.1-10.2B.11. May 1, 2001). The polyacrylamide gels were composed of 10% acrylamide, and the gels were run at 4° C. for 1 hour and 45 minutes. Samples of Taq only were run along with samples containing Reagent.

### Results

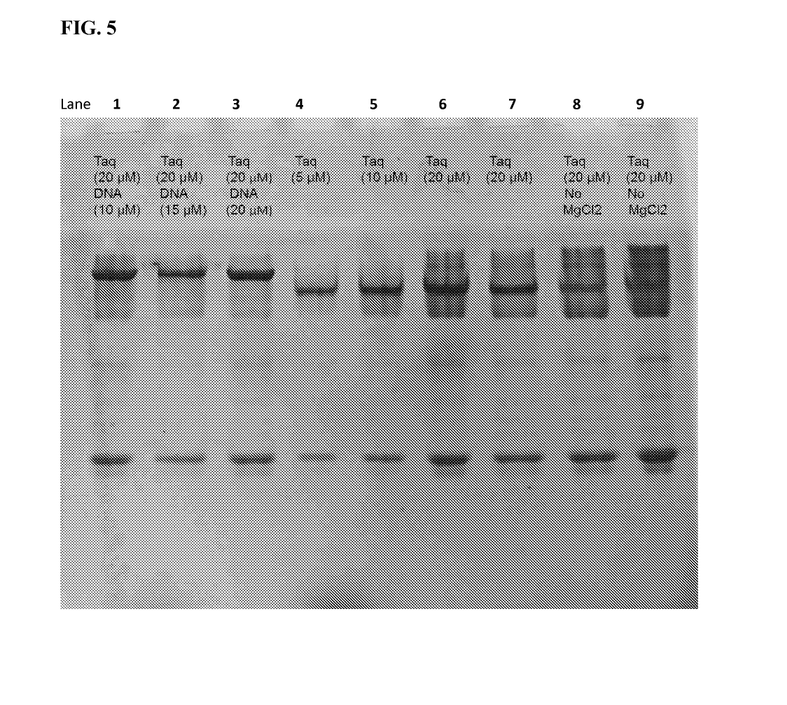

Results with the blocked dsDNA oligomer are shown in FIG. 5, lanes 1-9. The lanes all showed bands of a lower molecular weight than the Taq polymerase bands at the top of each lane. These bands were present on the SDS-PAGE gel. Lanes 4-7 of the Native gel are Taq alone at differing concentrations. Lanes 9-10 are Taq alone with no magnesium chloride. A strong Taq band is evident in the upper part of each lane, and a contaminant is evident in the lower part of each lane. As can be seen, the location of the Taq band was not affected by concentration or by the presence/absence of MgCl₂. Lanes 1-3 are samples containing 20 μM Taq and differing concentrations of the oligomer: 10 μM, 15 μM and 20 μM, respectively. Each of lanes 1-3 has a contaminant band corresponding to the band in lanes 4-9. Each of lanes 1-3 also has a strong band that migrated less than the Taq band in lanes 4-9. The Taq showed a clear shift in electrophoretic migration in response to the presence of dsDNA oligomer. The shift occurred with concentrations of the annealed oligomers even at molar ratios of 0.5:1 (10 μM oligomer:20 μM Taq) and 0.75:1 (15 μM oligomer:20 μM Taq). No other multimer states of Taq were detected. However, the lack of a band representing unbound Taq (despite the lack of DNA to bind to Taq in lanes 1-3) shows that the band represents dimeric Taq, not simply a monomer of Taq. FIG. 5 demonstrates that electrophoresis can be used to differentiate monomeric Taq from dimeric Taq. By recovering only a single band from lanes 1-3, only the dimeric Taq can be used as a composition according to this invention. We note that there were no molecular weight standards used in these assays. The reason for this is that molecular weight standards are typically used in denaturing gels, rather than in non-denaturing gels such as those used here. We found a single band of the Taq-and-DNA incubated samples.

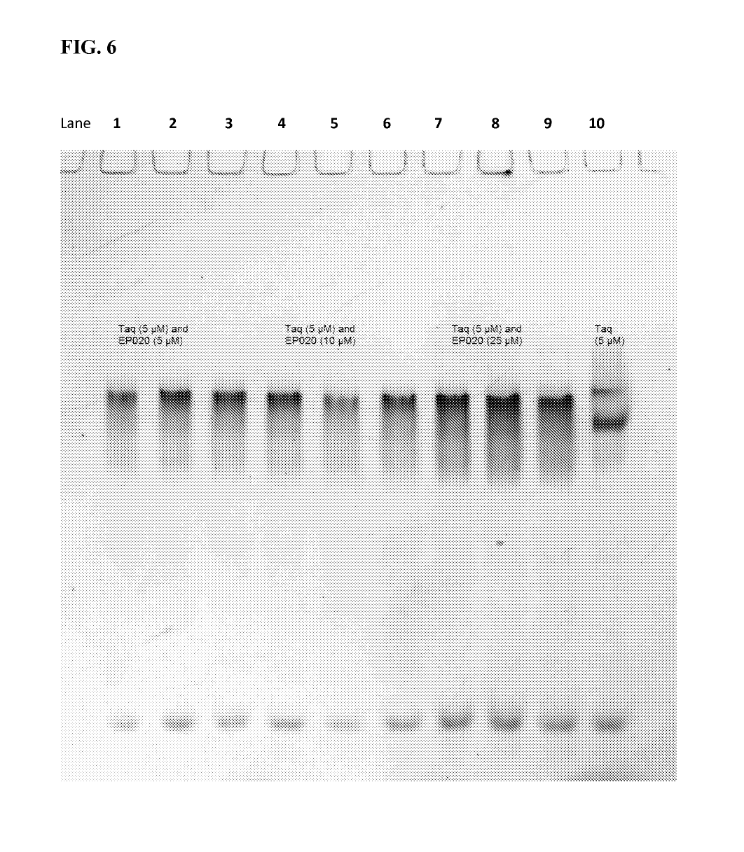

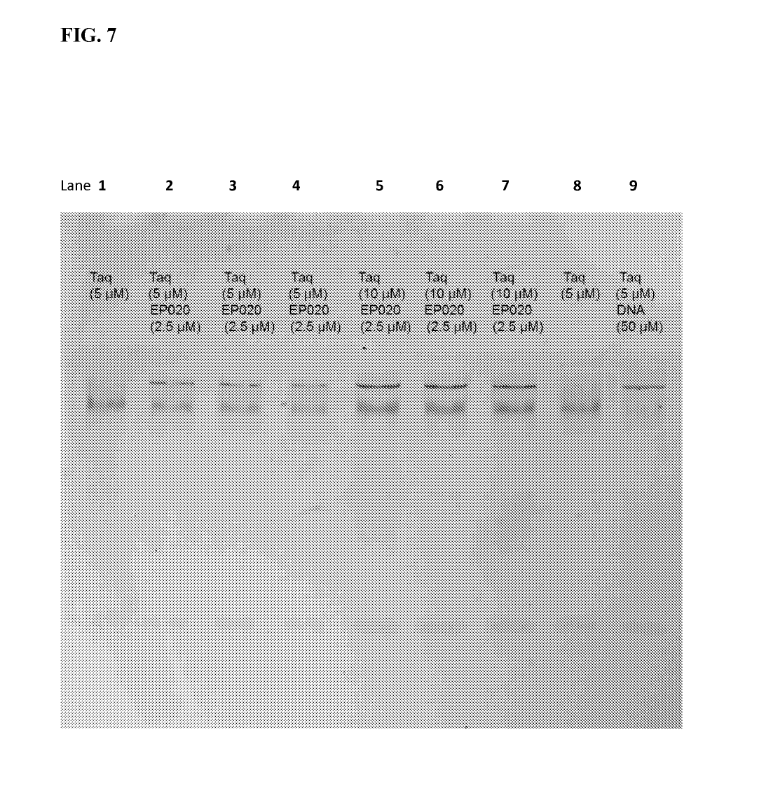

A similar set of tests were conducted with ratios of EP020 Type B Reagent and Taq. However, a more extensive range of EP020 was tested for binding to Taq. A first gel, FIG. 6, was run with 5 μM Taq (lanes 1-3), 10 μM (lanes 4-6) and 25 μM (lanes 7-9) EP020, as well as 5 μM Taq only (lane 10). In this test the concentration of Reagent equaled or exceeded the Taq concentration. A second gel, FIG. 7, was run with 5 μM Taq and 2.5 μM EP020 (lanes 2-4), 10 μM Taq and 2.5 μM EP020 (lanes 5-7), 5 μM Taq and 50 μM oligomer (lane 9), as well as 5 μM Taq only (lanes 1 and 8). The ratio of EP020 to Taq ranged from 10:1 (50 μM EP020 to 5 μM Taq) to 0.25:1 (2.5 μM EP020 to 10 μM Taq). The tests with the EP020 and Taq show a consistent binding event. The Taq/EP020 was shown to migrate more slowly than the unbound Taq. The amount of migration was the same for each ratio of EP020 to Taq in both FIG. 6 and FIG. 7. Referring to FIG. 6 and lane 9 of FIG. 7, the samples with a relatively high Reagent-to-Taq ratio, there was no monomeric Taq band, only a higher band of dimeric Taq. These results indicate a saturated binding event in the reactions. A saturation in binding is not surprising given the limited amount of Taq available.

Referring to FIG. 7, lanes 2-7, a second gel was run with EP020:Taq concentrations of 0.5:1 (2.5 μM EP020:5 μM Taq) and 0.25:1 (2.5 μM EP020:10 μM Taq). Taq-only was included (lanes 1, 8). Each of lanes 2-7 a monomeric Taq band, but each also included a band that is assumed to be dimeric Taq. The two observed bands correlate to the previously observed migrations of the two species in FIGS. 5 and 6. These tests showed that dimer formation is still possible in these conditions.

The results of these tests does not reveal the relative affinities of EP020 vs unlabeled DNA because the difference in length of the EP020 and the double-stranded DNA fragment used here, as well as the differences in their melting temperatures, may account for that. Lanes with EP020 and Taq showed three major bands.: an upper most band believed to be a complex of dimeric Taq and EP020; a second band whose migration equaled that of Taq without exposure to EP020, and was concluded to represent Taq without any other molecules bound; and a 3^{rd} band corresponding to one of the contaminants.

We claim:

1. A composition comprising a first Type I DNA polymerase molecule crosslinked to another molecule selected from the group consisting of a second Type I DNA polymerase molecule and a double-stranded reagent of Type A, Type B, Type C or Type D, said composition being free of unbound double-stranded reagent.

2. The composition of claim 1 comprising the first Type I DNA polymerase molecule crosslinked directly to the second Type I DNA polymerase molecule.

3. The composition of claim 1 comprising the first Type I DNA polymerase molecule crosslinked to the double-stranded reagent.

4. The composition of claim 3 wherein the first Type I DNA polymerase molecule and the second Type I DNA polymerase molecule are dimerized.

5. The composition of claim 4 wherein the first and second polymerase molecules are both crosslinked to the double-stranded reagent.

6. The composition of claim 1 wherein the composition exhibits enhanced polymerase selectivity compared to monomeric Type I polymerase.

7. A mixture of the composition of claim 1 and monomeric Type I polymerase.

8. A method of performing a DNA amplification reaction comprising the use of the mixture of claim 7.

9. The method according to claim 8, wherein the reaction is a PCR reaction.

10. The method according to claim 9, wherein the reaction is a LATE-PCR reaction.

11. A reaction mixture comprising a composition of claim 1 and amplification reagents for a PCR reaction, said reagents comprising primers and dNTPs.

12. A reagent kit comprising a composition of claim 1 and amplification reagents for a PCR amplification reaction, said reagents comprising primers and dNTPs.

13. The mixture of claim 7 comprising the first Type I DNA polymerase molecule crosslinked to the double-stranded reagent.

14. The mixture of claim 13 wherein the first Type I DNA polymerase molecule and the second Type I DNA polymerase molecule are dimerized.

15. The mixture of claim 14 wherein the first and second polymerase molecules are both crosslinked to the double-stranded reagent.

16. The mixture of claim 7 wherein the composition exhibits enhanced polymerase selectivity compared to monomeric Type I polymerase.

17. The mixture of claim 11 comprising the first Type I DNA polymerase molecule crosslinked to the double-stranded reagent.

18. The mixture of claim 17 wherein the first Type I DNA polymerase molecule and the second Type I DNA polymerase molecule are dimerized.

19. The mixture of claim 18 wherein the first and second polymerase molecules are both crosslinked to the double-stranded reagent.

20. The mixture of claim 11 wherein the composition exhibits enhanced polymerase selectivity compared to monomeric Type I polymerase.
